(19) United States
(12) Patent Application Publication
(10) Pub. No.: US 2010/0241001 A1
(43) Pub. Date: Sep. 23, 2010

(54) Ultrasound Methods, Systems and Computer Program Products for Imaging Fluids

(76) Inventors: Mark L. Palmeri, Durham, NC (US); David B. MacLeod, Durham, NC (US); Jeremy J. Dahl, Durham, NC (US); Stuart A. Grant, Chapel Hill, NC (US); Kathryn R. Nightingale, Durham, NC (US)

Correspondence Address: MYERS BIGEL SIBLEY & SAJOVEC, PO BOX 37428, RALEIGH, NC, 27627, US

(21) Appl. No.: 12/407,979

(22) Filed: Mar. 20, 2009

(51) Int. Cl. A61B 8/14

(52) U.S. Cl. 600/458

(57) ABSTRACT

Ultrasound systems for identifying a presence of injected fluid in a region of interest are provided. The ultrasound system includes a controller configured to obtain first and second image data sets of a region of interest from an ultrasound transducer array. A decorrelation module is configured to identify a decorrelation region of decorrelated data that is decorrelated between the first and second image data sets. The decorrelation region indicates a presence of injected fluid in the region of interest.

### FIELD OF THE INVENTION

The present invention relates to ultrasound methods, systems and computer program products, and more specifically to ultrasound imaging of fluids.

### BACKGROUND

Fluids injected in to the body can be difficult to image using conventional techniques. Such fluids can be anechoic and diffuse throughout the adjacent soft tissue during injection, which can result in obscuration of the structures of interest in ultrasound imaging.

The accurate delineation of injected anesthetics during regional nerve blocks could ensure that adequate nerve blockages are achieved because regional nerve blocks ideally require a substantially even distribution of anesthetic around the circumference of a target nerve/plexus. Accurate feedback of the distribution of injected anesthetic during injection can allow the anesthesiologist to reposition the needle to achieve the desired distribution. Anesthetic drugs may be ineffective if the distribution of the drugs around a target nerve is insufficient, which may result in intraoperative interventions, reduced post-operative pain control, and reduced post-operative function.

### SUMMARY OF EMBODIMENTS OF THE INVENTION

According to some embodiments, ultrasound systems for identifying a presence of injected fluid in a region of interest are provided. The ultrasound systems include a controller configured to obtain first and second image data sets of a region of interest from an ultrasound transducer array. A decorrelation module is configured to identify a decorrelation region of decorrelated data that is decorrelated between the first and second image data sets. The decorrelation region indicates a presence of injected fluid in the region of interest.

In some embodiments, the controller is configured to provide an image of the region of interest that identifies the decorrelation region. The decorrelation module can be configured to identify decorrelation data between the first and second image data sets based on a correlation coefficient (ρ) between a first signal, s₁(t), of the first image data set and a second signal, s₂(t), of the second data set as follows:

$$ρ = \frac{〈 {s}_{1} ( t ) {s}_{2}^{*} ( t ) 〉}{\sqrt{〈 {s}_{1} ( t ) {s}_{1}^{*} ( t ) 〉 〈 {s}_{2} ( t ) {s}_{2}^{*} ( t ) 〉}}$$

where * denotes the complex conjugate of the signal, ρ is a complex number whose magnitude denotes the similarity, or overall correlation, of the first and second signals and whose phase represents the phase difference between the first and second signals. In some embodiments, the decorrelation module is configured to identify decorrelation data such that ρ is less than about 0.98 and/or the complex signals s₁(t) and s₂(t), correspond to a region of image data for the first and second data sets, respectively.

In some embodiments, the decorrelation module is configured to identify decorrelation data between three or more image data sets, and the controller is configured to provide an image of the region of interest that identifies combined decorrelation data from three or more image data sets.

According to some embodiments, ultrasound methods for identifying a presence of injected fluid in a region of interest include obtaining first and second image data sets of a region of interest from an ultrasound transducer array. A decorrelation region of decorrelated data is identified that is decorrelated between the first and second image data sets. The decorrelation region indicates a presence of injected fluid in the region of interest.

In some embodiments, an image of the region of interest is provided that identifies the decorrelation region. A fluid can be injected in the region of interest, and an injection location can be modified based on the image of the region of interest and the identified decorrelation region.

According to some embodiments, a computer program product for identifying a presence of injected fluid in a region of interest includes a computer readable medium having computer readable program code embodied therein. The computer readable program code includes computer readable program code configured to obtain first and second image data sets of a region of interest from an ultrasound transducer array. Computer readable program code is configured to identify a decorrelation region of decorrelated data that is decorrelated between the first and second image data sets. The decorrelation region indicates a presence of injected fluid in the region of interest.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a schematic drawing of an ultrasound system according to some embodiments of the present invention.

FIG. 2 is a flowchart illustrating operations according to embodiments of the present invention.

FIG. 3 illustrates digital images of an in vivo B-mode ultrasound image (a) and an Acoustic Radiation Force Image (ARFI) (b) of the tibeal and common peroneal nerves in a 29 year old subject just distal to their bifurcation from the sciatic nerve in the popliteal fossa. The ARFI image (b) was generated using two excitation focal zones at 15 and 20 mm with a VF7-3 linear array. The peak displacement in the ARFI image is 4 μm. ECG data acquisition gating was not used in generating the ARFI image. The two nerves are clearly delineated and are stiffer (darker) circular structures in cross-section; their location is not readily apparent in the B-mode image (they have been outlined in square cross-hatching (tibial) and diagonal cross-hatching (popliteal) based on the ARFI image boundaries). The improvement in nerve contrast was over 600% in the ARFI image.

FIG. 4 illustrates digital images of an in vivo B-mode ultrasound image (a) and an Acoustic Radiation Force Image (ARFI) (b) of the rectus abdominus muscles and rectus sheath in a 29 year old subject generated with a VF10-5 linear array. The posterior sheath is located at a depth of 14-16 mm in these images, and its anterior surface is the target for local anesthetic injection. The ARFI image highlights the greater mechanical contrast that exists in this targeted layer compared with the more superficial fascial planes (i.e., it is darker, representing greater relative stiffness, compared to the adjacent muscle and superficial fascia). Given the higher overall stiffness of these tissues compared with the structures shown in FIG. 3, the peak displacement in the ARFI image is 1 μm.

FIG. 5 illustrates digital images of in vivo B-mode ultrasound images (a) (first column) and Acoustic Radiation Force Images (ARFI) (b) (second column). An 18 gauge needle aligned parallel to the transducer face, inserted from the left of the image with the tip near a lateral position of 0. Imaging was performed with the needle (top row) centered in the imaging plane, (middle row) 2 mm (3.2× the needle's radius) offset from the center of the imaging plane in elevation, and (bottom row) 5 mm (8× the needle's radius) offset from the center of the imaging plane in elevation. This imaging was performed in beef muscle using a VF7-3 linear array using 3 excitation focal zones (18, 22, and 26 mm). The needle quickly disappears from view in the B-mode images with slight misalignment, but is still visible as a stiff (dark) line on the corresponding ARFI images.

FIG. 6 illustrates digital images of B-mode images (top row) and ARFI images (bottom row). Column (a) illustrates an 18 gauge needle centered in the imaging plane at a 50° angle relative to the VF7-3 transducer face. While the needle is not easily visualized in the B-mode image, it is clearly visible in the ARFI image, though there is significant decorrelation below the needle due to the low SNR. Column (b) illustrates an 18 gauge needle parallel to the transducer face in cross-section at a depth of 18 mm. The needle itself shows up as a black decorrelated circle with decorrelation deep to it. There is also a ring of stiffened tissue around the needle extending an additional 2 mm from the needle's edges. This stiffening of tissue adjacent to the needle is what allows visualization of the needles being imaged on length when they are slightly mis-aligned with the imaging plane, as shown in FIG. 5.

FIG. 7 illustrates digital images of B-mode images (top row) and ARFI images (bottom row) of a 27 gauge needle nearly parallel to the transducer (VF7-3) face with its tip at a lateral position of about 1 mm. Image (a) was centered in the imaging place and (b) was 1 mm offset from the center of the imaging plane. The tissue surrounding the needle is still stiffer (darker) with the 1 mm offset (5× greater than the needles radius) extending to the needle's tip.

FIG. 8 illustrates B-mode and ARFI digital images of an in situ distal cadaveric sciatic nerve with an 18 gauge needle intentionally piercing the nerve sheath in the nerve's upper left quadrant, as imaged with the VF7-3 linear array. There is significant decorrelation deep to the needle due to poor SNR, but the tip of the needle and the edge of that decorrelation can be see inside the left border of the sciatic nerve, which appears dark (stiff) in the ARFI image.

FIG. 9 illustrates B-mode and ARFI images generated with the VF10-5 linear array of the brachial plexus in vivo from an intersealene approach pre-injection (top row), after a 2 cc saline injection (middle row), and after an additional 4 cc saline injection, for a total of 6 cc saline injected into that site (bottom row). Preinjection (top row), the needle (top arrows) was placed adjacent to the lower left aspect of the nerve bundle (bottom arrows). The substructures of the nerve bundle spread apart and are displaced to the right after the saline injections. Some of the nerve substructures have displacement over 5 mm laterally in response to the injection. This is more apparent in the ARFI images than the corresponding B-mode images. The contrast improvement in the nerve was almost 300% in the ARFI image compared with the B-mode image.

FIG. 10 illustrates in vivo injection of saline near the brachial plexus through an interscalene approach imaged with the VF10-5 linear array. Regions of decorrelation (ρ<0.98) are shown in cross-hatching. The area of the decorrelated region is roughly twice as large after 4 cc of saline have been injected (column c) compared with 2 cc of injected saline (column b). The needle can be seen approaching the nerve from left at a depth of 8-10 mm. The nerve in this example is brighter than the adjacent soft tissue (muscle), indicating that it is actually softer than the adjacent muscle. This highlights the fact that ARFI images depict relative stiffness, and absolute stiffness values cannot be based on displacement magnitudes alone. Also note that these decorrelation masks can be made without the presence of an radiation force excitation (middle row), but can also be combined with a standard ARFI image to more clearly delineate the adjacent needle and nerve.

FIG. 11 illustrate digital images of a single ARFI excitation (a), a 2D ARFI image sequence (b), and a graph of the normalized temperature as a function of time (c) for an exemplary thermal simulation for the VF10-5 linear array operating at 6.7 MHz in a homogeneous material (α=1.0 dB/cm/MHz) that used focal configurations and thermal material properties appropriate for imaging breast tissue. The heating (° C.) immediately after a single ARFI excitation (a) closely represents the transmitted acoustic field, while an ensemble of excitations to generate a 2D image (b) creates a more distributed heating field with translation of the peak heating location into the near field away from the focal point. The simulation results also predict cooling due to thermal diffusion that agrees well with Type-T thermocouple measurements (c), allowing a design of ARFI imaging beam sequences to minimize soft tissue heating.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, embodiments of the present invention may take the form of a computer program product on a computer-usable or computer-readable storage medium having computer-usable or computer-readable program code embodied in the medium for use by or in connection with an instruction execution system.

The computer-usable or computer-readable medium may be, for example but not limited to, an electronic, magnetic, optical, electromagnetic, infrared, or semiconductor system, apparatus or device. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM).

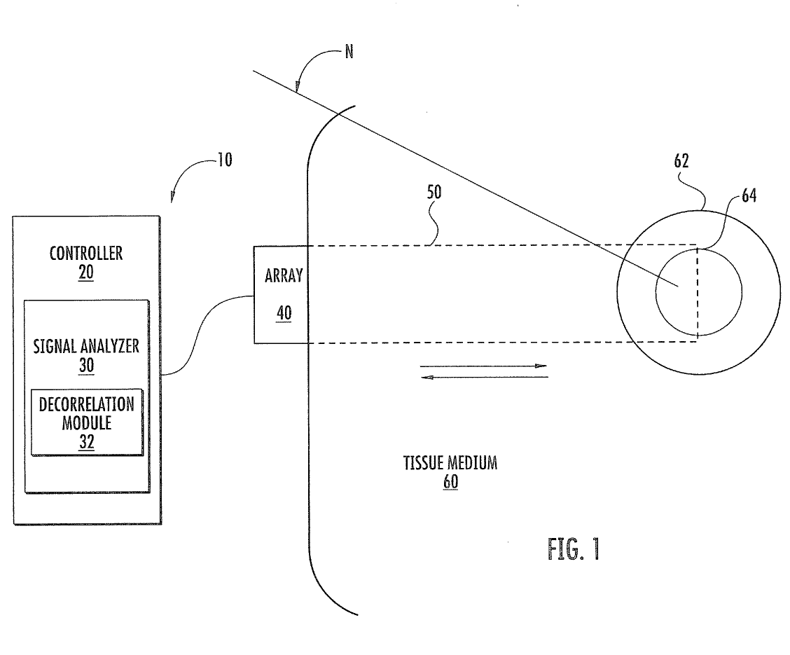

As illustrated in FIG. 1, an ultrasound system 10 includes a controller 20, a signal analyzer 30 and an ultrasound transducer array 40. The ultrasound transducer array 40 is configured to transmit and receive ultrasound signals 50, and may be contacted to a target medium such as a tissue medium 60. As illustrated, the tissue medium 60 includes a target region 62 having an injection region 64.

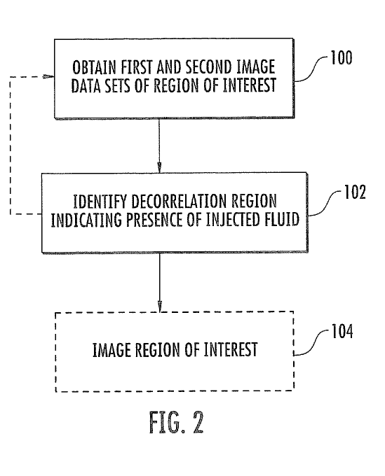

In some embodiments, the ultrasound system 10 is configured to identify a presence of an injected fluid from a needle N in the injection region 64. With reference to FIGS. 1 and 2 the signal analyzer 30 can acquire at least two image data sets of the region 62 from the ultrasound transducer array 40 (FIG. 2, Block 100). The signal analyzer 30 can include a decorrelation module 32, and the decorrelation module 32 can be configured to identify decorrelated data that are decorrelated between the two image data sets (FIG. 2, Block 102). The decorrelation region generally indicates a presence of injected fluid in the target region 62.

Without wishing to be bound by theory, fluid flowing from the needle N and injected into the tissue medium 60 can cause movement of the tissue and/or other changes in tissue composition in the injection region 64 that can be detected by decorrelated data. Thus, the decorrelation of ultrasound data between images that are spaced apart by some period of time (e.g., 0.1 ms) can indicate the presence of injected fluid within the decorrelation region. Accordingly, the identification of decorrelated data between image sets can be used to approximate and/or image the region in which injected fluids are present. In some embodiments, the controller 20 is configured to provide an image of the tissue medium 60 that identifies the decorrelation region (FIG. 2, Block 104). For example, an image can be provided in which the decorrelation region is shown in a contrasting color. In some embodiments, the ultrasound images described herein can be used to provide substantially real-time feedback during injection procedures. The image can include a target nerve and an approximation of the distribution of injected fluid, such as an anesthetic, substantially in real-time during injection. A health care professional can view the image to ensure that a desired distribution of anesthetic is achieved around the targeted nerve. In some embodiments, the health care professional can reposition the needle to achieve the desired distribution. Accordingly, embodiments of the present invention can be used to reduce incidences of failed nerve blocks.

As illustrated in FIG. 2, in some embodiments, additional image data sets are obtained (Block 100) and the step of identifying a decorrelation region based on decorrelation between the image data sets is repeated (Block 102). Accordingly, decorrelation data can be obtained from a plurality of three or more image data sets. In some embodiments, an image of the decorrelation region in the region of interest (Block 104) identifies a region including all of the decorrelation regions from the plurality of image data sets.

Although embodiments of the current invention are described herein with respect to injected fluids for use in anesthetic procedures, it should be understood that embodiments of the current invention can be applied to other procedures using injected fluids, such as amniotic fluid injections in obstetrics and corticosteroid injection in orthopedic surgery and/or sports medicine.

Moreover, embodiments of the current invention can be used with conventional B-mode ultrasound imaging data and/or acoustic radiation force imaging (ARFI) data. For example, the controller 20 and ultrasound array 40 can be configured to obtain conventional B-mode images or ARFI images in which the array 40 emits a series of low intensity “tracking lines” and higher intensity “pushing” pulses to interrogate the tissue medium 60. Various ultrasound techniques are described, for example, in U.S. Pat. Nos. 7,374,538 and 6,371,912, the disclosures of which are hereby incorporated by reference in their entireties. In some embodiments, B-mode and ARFI imaging data can be combined to provide a single image, and the decorrelation region or decorrelation map can be identified on the combined image. Moreover, two- or three-dimensional images can be used. It should also be understood that the ultrasound array 40 can be a one- or two-dimensional array having various numbers of ultrasound array elements.

Embodiments according to the present invention will now be described with respect to the following non-limiting examples.

### EXAMPLES

The software on a Siemens SONOLINE ANTARES™ scanner platform (Siemens Ultrasound Division, Mountain View, Calif., USA) is modified to realize ARFI-imaging capabilities. ARFI imaging on a wide range of phased, linear, curvilinear, and intracardiac probes with frequencies ranging from 2 to 10 MHz, each with 192 elements and 192 simultaneously active transmit and receive channels have been developed. This system can acquire and store the raw radio-frequency (RF) or in-phase/quadrature (I/Q) signals to produce concurrent ARFI and B-mode images synchronized with an ECG signal. Multiple excitation focal zones can be utilized to generate an ARFI image to improve structural contrast. The RF or I/Q data can be passed to a processing node via an Ethernet connection, and the displacements estimated and the resulting images displayed within a few seconds.

The ARFI images described herein utilize a grey scale map, where brighter pixels indicate greater displacement (i.e., more compliant tissue) compared with darker pixels (i.e., stiffer tissue). ARFI images represent the spatial average of the displacement fields resulting from 2-3 excitation focal zones, usually spaced 3-5 mm apart from one another axially. The lateral spacing for each focal zone is typically 0.15 mm. Each focal zone is interrogated sequentially (for 6.5 ms), thus for a typical sequence, the total image acquisition time is: (160 lateral positions/4 lateral positions per excitation)*3 focal zones*6.5 ms=0.78 seconds, without pulse interleaving between spatially-offset positions. The settings can be controlled/modified, and thus typical parameters are provided herein. In each excitation location, a pulse sequence ensemble typically includes: three reference ‘A-lines,’ followed by a high intensity pushing beam, followed by a series of ‘A-line’ tracking lines, typically at a PRF of 8 kHz, for 6.5 ms. The tracking lines are typical B-mode lines: frequency in the upper bandwidth of the transducer, 1 cycle, F/0.5 dynamic receive focal configuration. The pushing beams typically utilize center frequencies in the lower end of the transducer bandwidth, <100 μs in duration, Isppa_{.5} of about 3000 W/cm² (derated, linearly extrapolated small signal measurement in water), MI of about 2.3. Displacements are computed using I/Q data. Typical peak displacement magnitudes in the following images are 2-5 μm. When present, pixels on the ARFI images can be identified, e.g., using color contrast, to indicate regions of decorrelation determined during displacement estimation, with decorrelation being defined as |ρ|<0.98 (Equation 1, below). Other values of ρ may be used, such as between 0.5 and 0.9. All temporal displacement data were motion filtered assuming a linear motion artifact over the temporal tracking domains (about 6.5 ms). Unless otherwise noted, ARFI images represent the synthesized displacement response 0.55 ms after cessation of each radiation force excitation.

ARFI Imaging of Nerves

In many regional anesthesia cases, nerves can be difficult to visualize due to their similar acoustic impedance with surrounding tissues, including fat, muscle, and connective tissue. This can create limitations in the utility of B-mode imaging to delineate these structures for injection image guidance.

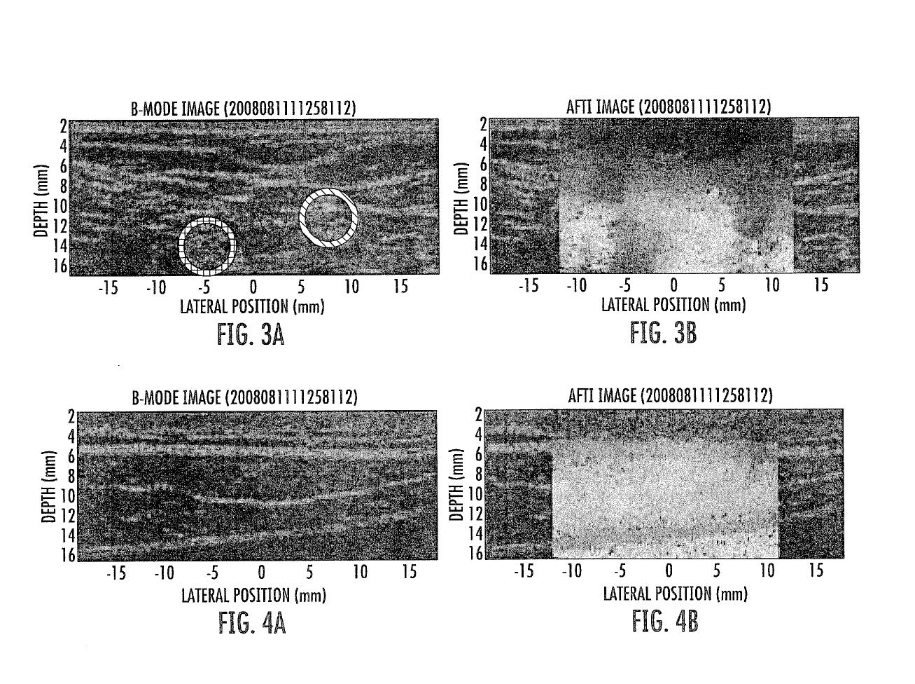

ARFI imaging does not generally rely on these acoustic impedance mismatches, and instead can generate images of displacement that are indicative of the localized stiffness properties of the tissue. FIG. 3 shows in vivo B-mode and ARFI images of the tibeal and common peroneal nerves, just distal to their bifurcation from the sciatic nerve in the popliteal fossa in a 29 year old subject. The nerves may be very difficult to appreciate in the B-mode image in FIG. 3, but the nerves are clearly delineated in cross-section as dark circular entities in the ARFI image. FIG. 3 demonstrates the potential clinical utility of using ARFI images to identify nerve anatomy in vivo.

The visualization of fascial planes can provide very useful anatomical feedback for anesthesiologists when performing injections. For example, in a rectus sheath block (used for umbilical and midline surgical procedures), there are several fascial planes that can be visualized in B-mode imaging. The target for the local anesthetic is the terminal branches of the intercostal nerves in the rectus sheath (in the space posterior to rectus abdominus muscle and the posterior sheath). As shown in FIG. 4, these tissue planes are visible in the B-mode image, but the ARFI image provides mechanical distinction between the different fascial planes. The fascia between the rectus abdominus muscles has much less mechanical contrast compared with the posterior sheath, which is much darker in the ARFI image, indicating that it is relatively stiffer.

Comparison With Elastography

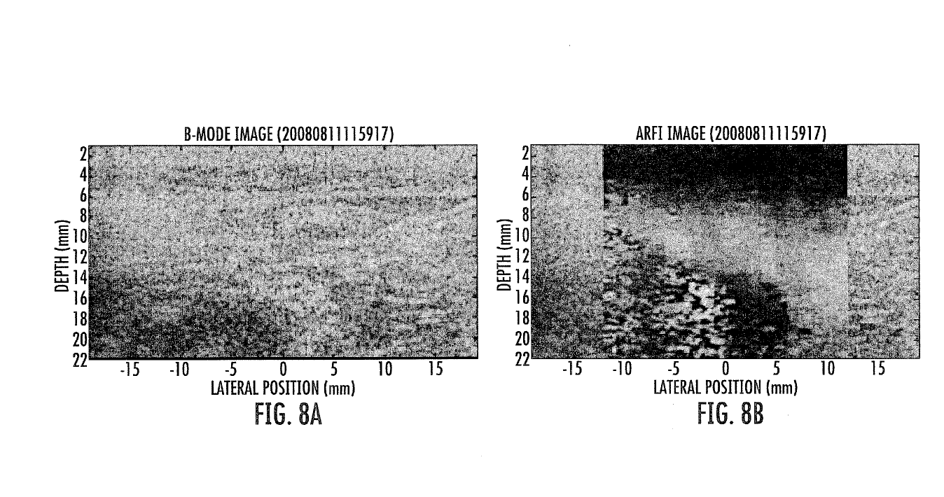

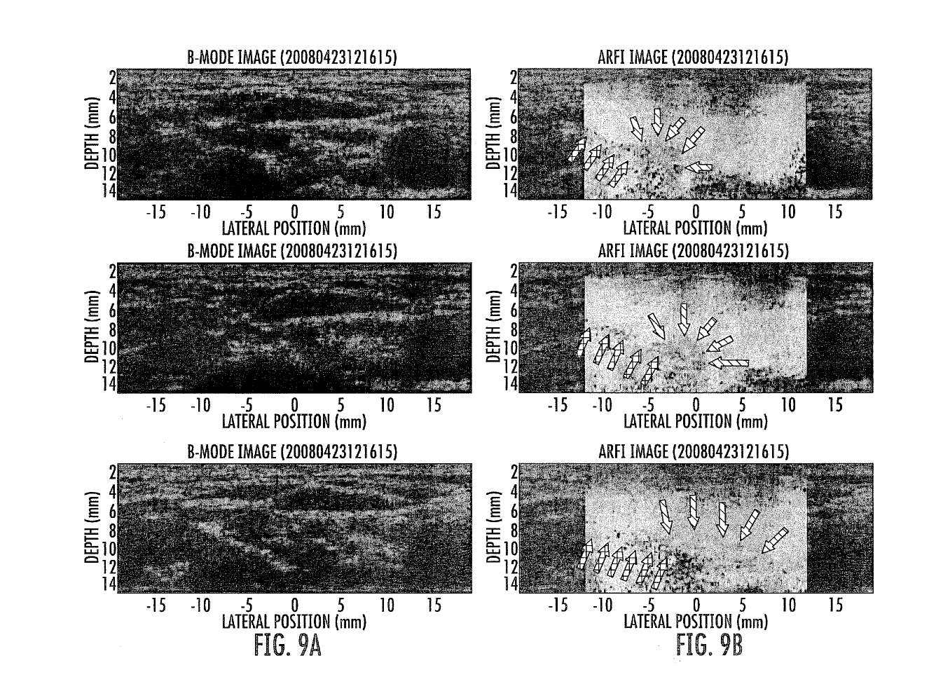

Elastography has been implemented on several commercial scanners, including the ACUSON ANTARES™ scanner (Siemens Ultrasound Division, Mountain View, Calif., USA). During preliminary studies with ARFI imaging of nerves, the Antares elastography mode was to provide some of the imaging improvements also being targeted with ARFI imaging. Images of the brachial plexus and sciatic nerves were acquired using a VF7-3 linear array operating at 7.3 MHz. The elastography images exhibited relatively high noise. Elastography can rely on a uniform stress to be applied to the ROI to generate strain images that are directly reflective of the underlying soft tissue stiffness, and therefore, compression with a hand-held transducer and physiologic motion near arteries make uniform stress conditions difficult to achieve. These potential problems with elastography may be further complicated with the introduction of a needle in the region of interest. Commercial implementations of elastography may not be able to achieve the improvements in nerve visualization/contrast that are present in ARFI images (FIGS. 3, 8 and 9).

ARFI Imaging of Needles

Needles may be clearly delineated in B-mode images when they are (1) at shallow angles of approach relative to the transducer face (near parallel to the transducer face) and (2) well-aligned with the imaging plane. The “thickness” of an imaging plane in the depth of field is <1 mm for a standard linear array focused at 20 mm, operating at 7 MHz, with an F/4 focal configuration in the elevation dimension. This small imaging plane thickness can make alignment with the needle very challenging. When at steep insertion angles, needles are also difficult to visualize since most of the acoustic energy they reflect is directed away from the receive aperture of the transducer, making them almost acoustically invisible. The presence of a needle causes a stiffening of the adjacent tissue that it is hypothesized can be visualized with ARFI imaging (1) when the needle is slightly misaligned with the imaging plane, and (2) when the needle is at steep insertion angles relative to the transducer face. Being able to visualize a needle in these orientations may allow an anesthesiologist to make well-placed injections. A series of experiments were performed in ex vivo beef muscle using standard 18 gauge (1.27 mm outer diameter) and 27 gauge (0.406 mm outer diameter) needles to evaluate how far out of the imaging plane needles could be appreciated in ARFI images and how needles appeared in ARFI images when at steep angles and in cross-section.

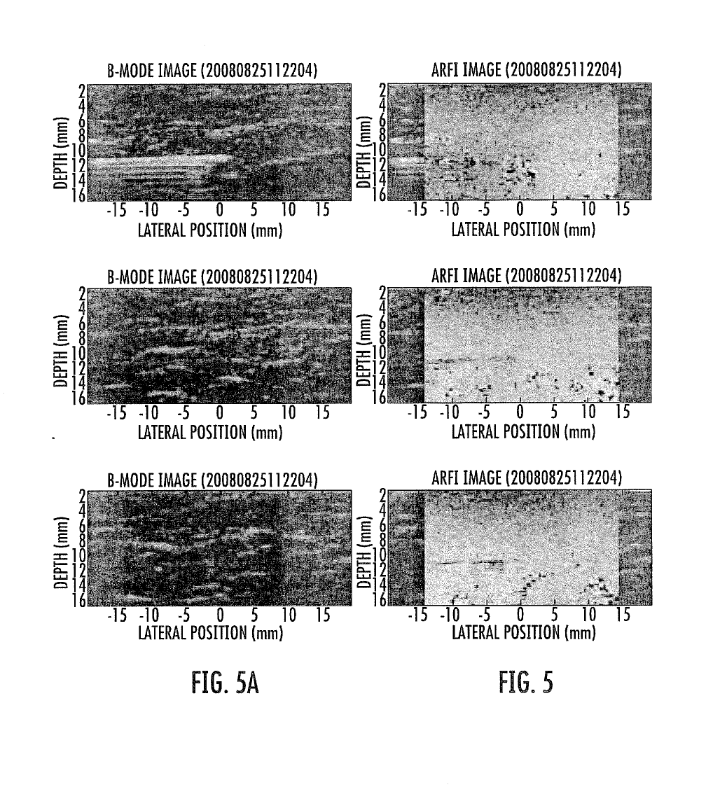

FIG. 5 shows an 18 gauge needle imaged (top row) directly aligned in the imaging plane, (middle row) 2 mm offset from the imaging plane in the elevation dimension, and (bottom row) 5 mm offset from the imaging plane in the elevation dimension. The needle was aligned parallel to the transducer face for all images. Needles can be much more difficult to visualize in B-mode images when the angle of approach relative to the transducer face increases as more sound is reflected away from the transducer.

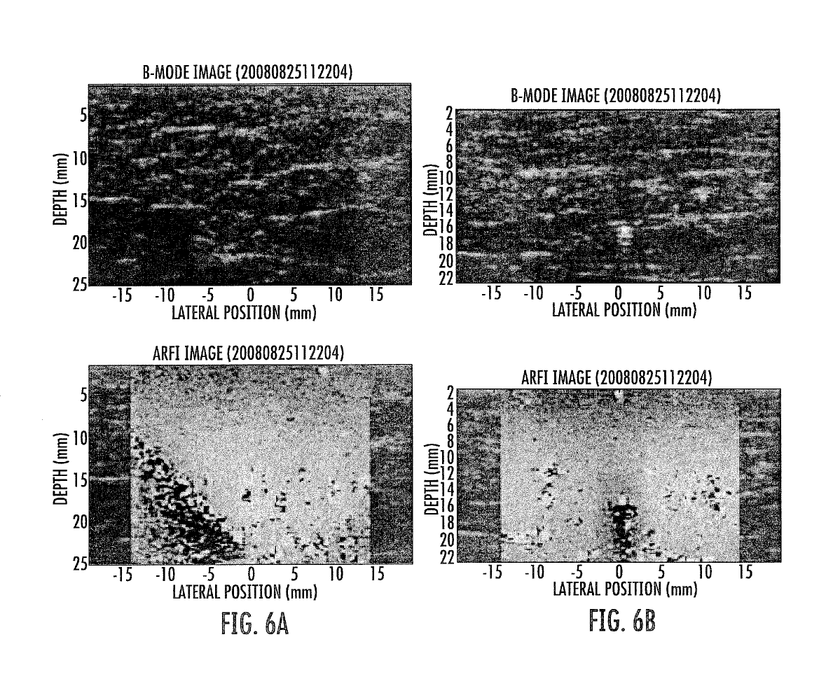

FIG. 6, column (a) shows B-mode and ARFI images of an 18 gauge needle at a 50° angle relative to the transducer face. FIG. 6, column (b) shows the same needle re-aligned parallel to the transducer face at a depth of 18 mm in cross-section. Needles can be difficult to visualize in cross-section because the hyperechoic reflections are small and can be mistaken for other small hyperechoic structures. The hyperechoic signatures of needles in cross-section in B-mode images are also reduced when the needle is at an angle relative to the transducer face.

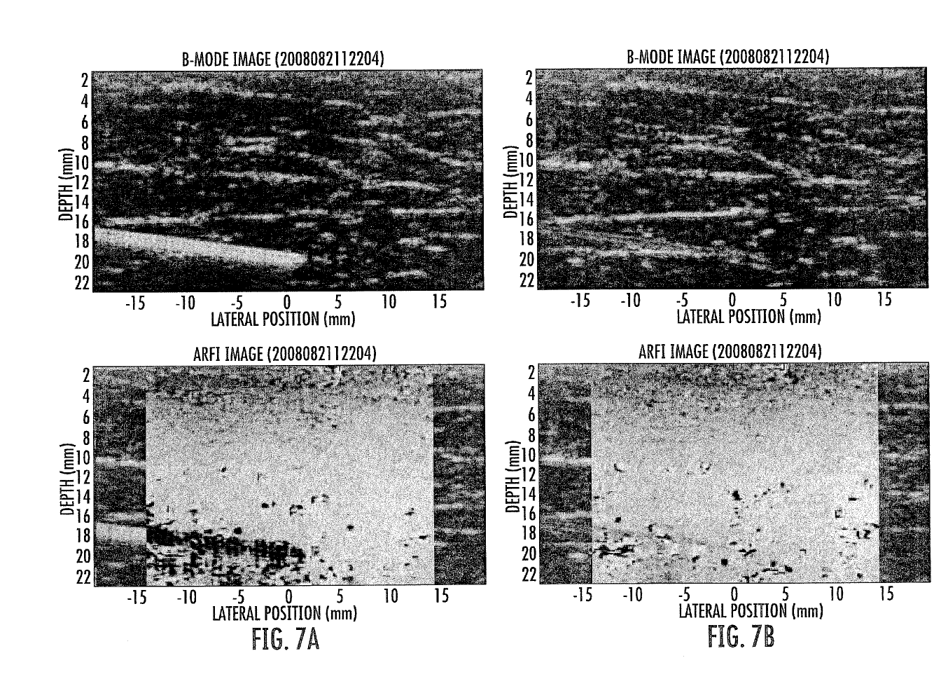

While 18 gauge needles are commonly used for regional nerve block procedures, needles as small as 25 gauge can be used for certain anatomical locations (e.g., high brachial plexus blocks). FIG. 7 demonstrates the ability to use ARFI imaging to see a needle as small as 27 gauge with a 1 mm offset from the center of the imaging plane.

FIG. 7 shows how ARFI imaging can be used to visualize needles and nerves concurrently in a cadaveric distal sciatic nerve in situ. An 18 gauge needle was inserted into the left upper quadrant of the nerve. The needle causes significant reflection of the ultrasound energy, leading to poor signal-to-noise ratio (SNR) deep to the needle that is very apparent in the ARFI image. The ARFI image can delineate the tip of the needle inside the left border of the sciatic nerve, which appears dark (stiff) in the ARFI image.

The contrast improvement in the nerve was almost 300% in the ARFI image compared with the B-mode image.

In summary, ARFI images are able to delineate needles at steep angles of approach and needles misaligned with the imaging plane that may not otherwise be visible in matched B-mode images (FIGS. 5-8).

ARFI Imaging of Injections

The soft tissues surrounding a nerve are infused with local anesthetic when an injection is performed.

The injectate is anechoic and can cause significant distortion of the pre-injection B-mode image, making it difficult to evaluate where the anesthetic agent is distributing relative to the nerve of interest. The nerve itself may be displaced from the ROI and may have its structure distorted by the injection. Knowledge of this motion/distortion of the nerve may be useful to an anesthesiologist so that the needle could be repositioned to achieve a more effective nerve block where the entire circumference of the nerve is uniformly bathed with local anesthetic. FIG. 9 shows how the nerve of interest (in vivo brachial plexus from an interscalene approach) can be translated and distorted after a 2 cc saline injection (middle row) followed by an additional 4 cc saline injection (bottom row). These injected volumes represent a small fraction of the total volume of local anesthetic (about 30 cc) that can be injected for a nerve block.

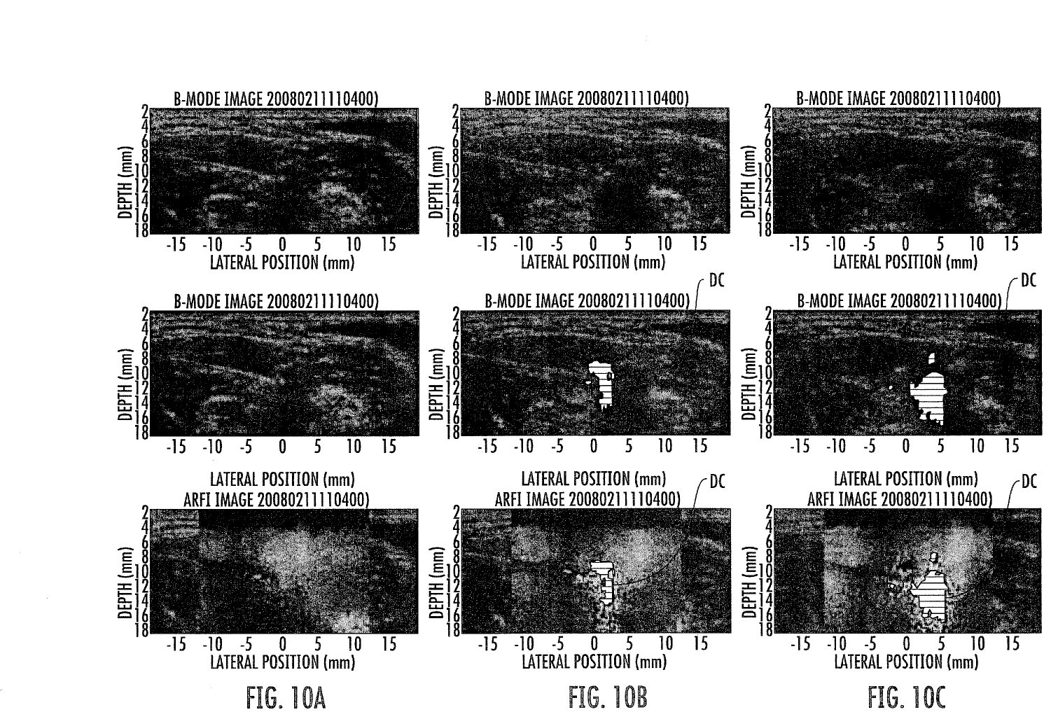

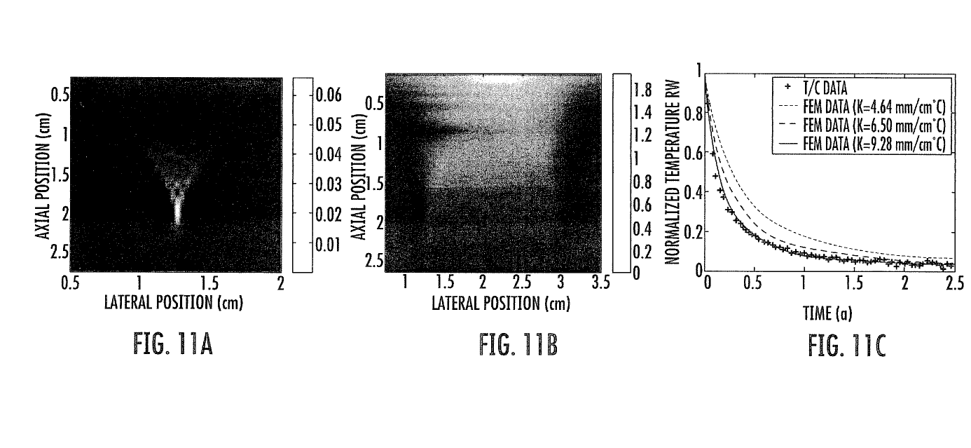

While the changes in nerve position in FIG. 9 are indirectly representative of the distribution of the adjacent anesthetic, the nerve's position can also be dictated by out-of-plane structures. A more direct visualization of the distribution of the injectate may aid an anesthesiologist in determining how evenly a nerve is being surrounded with anesthetic. Since the anesthetic is causing a disruption of the preinjection B-mode speckle, there is a decorrelation between successive imaging frames as more anesthetic is injected. FIG. 10 show regions of decorrelation (ρ<0.98, as defined by Equation 1) in cross-hatching that occur after a 2 cc injection of saline adjacent to the brachial plexus through an interscalene approach (column b) and after an additional 2 cc of saline is injected (column c). Note that these decorrelation maps can be made in the absence of a radiation force excitation, as shown in the middle row, or can be made concurrently with ARFI images to provide better visualization of the adjacent needle and nerve. The accuracy of the spatial delineation of injection distribution relative to the visualized nerves and needles will be characterized in cadaveric specimens with methylene blue injections. The nerve in this location is brighter than the adjacent soft tissue (muscle), indicating that it is actually softer than the adjacent muscle. This highlights the fact that ARFI images depict relative stiffness, and absolute stiffness values cannot be based on displacement magnitudes alone.

Signal Decorrelation Quantification:

Loupas' method is used on I/Q data to estimate tissue displacements. The phase-shift approach to displacement estimation is also amenable to calculation of a complex correlation coefficient. The complex correlation coefficient between two complex signals s₁(t) and s₂(t) is computed as follows:

$$ρ = \frac{〈 {s}_{1} ( t ) {s}_{2}^{*} ( t ) 〉}{\sqrt{〈 {s}_{1} ( t ) {s}_{1}^{*} ( t ) 〉 〈 {s}_{2} ( t ) {s}_{2}^{*} ( t ) 〉}} \qquad ( 1 )$$

where * denotes the complex conjugate of the signal, ρ is a complex number whose magnitude denotes the similarity, or overall correlation, of the two complex signals and whose phase represents the phase difference between the two signals. For example, a magnitude of 1 and a phase of 0° indicates perfect correlation, while a magnitude of 1 and a phase of 180° indicates negative correlation. Assuming ARFI induced displacements are in the direction of wave propagation (away from the transducer), the phase varies between 0-90°, and the magnitude of this complex coefficient represents the correlation between tracking vectors. This magnitude was used to generate the decorrelation maps in the ARFI images of injections above. However, it should be understood that various techniques and/or robust methods for decorrelation mapping of the distribution of injected anesthetic under varying imaging conditions can be provided. The complex signals s₁(t) and s₂(t) can correspond to an image region of decorrelation, such as a pixel or group of pixels.

Although embodiments according to the present invention are described herein with respect to complex correlation coefficients, it should be understood that radio frequency (RF) data and real correlation coefficients can also be used, for example, using the complex correlation coefficient expression such that the complex operations are applied exclusively to the real components. Any number of phase-shift estimators can be used when processing I/Q data (e.g., Loupas's and Kasai's method), while RF data can be processed using correlation-based algorithms (e.g., normalized cross correlation). Regardless of the displacement estimation algorithm, a correlation coefficient (real or complex) can be computed to determine the amount of decorrelation between two signals.

Safety of Radiation Force Imaging

Thermal Bioeffects

The FDA currently considers temperature rises less than 6° C. in soft tissue (excluding obstetric and optical imaging) during diagnostic ultrasound imaging to be acceptable. Simulation tools have been developed to investigate the tissue heating associated with ARFI imaging sequences. These simulation tools can predict the three-dimensional heating that occurs in response to a single (FIG. 11(a)) or ensemble (FIG. 11(b)) of ARFI imaging insonifications, and solves the linear bioheat transfer equation (Equation 2) to determine the cooling time constants (FIG. 11(c)) associated with thermal diffusion in soft tissue. Perfusion is neglected in this analysis to provide a conservative cooling estimate.

$$\dot{T} = κ {∇}^{2} T + \frac{{q}_{v}}{{c}_{v}} \qquad ( 2 )$$

In Equation 2, T[° C.] is the temperature, \dot{T}[° C./s] is the time rate change in temperature, κ[0.00143 cm2/s] is the thermal diffusivity for soft tissue, qᵥ[J/cm3] is the rate of heat production per unit volume, and cᵥ[4200 mW·s/cm3/° C.] is the heat capacity per unit volume for soft tissue. The value qᵥ can be estimated by 2I, where I[W/cm²] is the acoustic beam intensity. The derated spatial peak pulse average intensities that are realized for an ARFI excitation pulse are comparable to those used for High Intensity Focused Ultrasound (HIFU), on the order of 1500 W/cm² (Isppa.5) if measured with a hydrophone in a water-tank. It has been found, however, that derated water-tank measurements considerably underestimate the in situ intensities due to nonlinearity and saturation effects in water, by approximately 50%. As a result, the approach of derating linearly extrapolated small signal values is taken to determine input acoustic intensities to scale the simulations, and with this approach good agreement between simulated results and experimental measurements of both thermal and mechanical measurements are obtained.

ARFI imaging and HIFU pulses differ primarily in their duration. ARFI excitation pulses are applied for only about 100 μs, as compared to about 5 seconds for HIFU. The typical temperature rise associated with each ARFI excitation is <0.2° C., and this peak heating occurs near the focus (at the location of the peak energy in the ROE). With the use of 4:1 parallel receive processing to monitor displacements in ARFI imaging, 1:4 the number of ARFI excitations are fired for the number of lateral locations used to form an ARFI image, reducing the amount of heat generated in the tissue by a factor of 4. As with HIFU, spatially translating the excitation beams throughout a 2D field of view (FOV), as is done in 2D ARFI sequences, shifts the location of peak heating into the near-field to the location of maximum energy deposition. Tissue temperature increases within ultrasonic diagnostic levels using 2D ARFI sequences over a fairly large FOV (e.g., 2.5 cm laterally) for a single frame acquisition. However, it is critical to simulate and experimentally characterize the tissue heating with thermocouples over a range of anticipated tissue parameters during sequence development to ensure that the maximum tissue heating will not exceed a threshold. While the AIUM recently proposed more liberal thermal limits for insonification in adults, all of the clinically-implemented beam sequences herein are designed to not exceed ½ of the FDA limit (i.e., the maximum tissue temperature will not exceed 3° C.). IRB approval has been obtained for several ARFI imaging protocols in different organ systems (e.g., blood vessels, livers, muscles, and kidneys), and no adverse affects have been experienced during any ARFI imaging protocols. As with other imaging protocols, ARFI images can avoid having bone in the ROE to avoid foci of increased heating.

The presence of a needle in the proposed studies will change the heating patterns that have typically been simulated with the assumptions of a purely absorptive medium. The Finite Element Method (FEM) simulations will be modified to accommodate the presence of a needle at an arbitrary angle relative to the transducer face to evaluate the spatial distribution of the thermal energy associated with this reflective interface. These findings will be experimentally validated in tissue-mimicking phantoms using embedded thermocouples. Given the original conservative heating limits of ½ of the FDA limit of 6° C., it is expected to remain well within the safety limits with the presence of a needle in the ROE.

Non-Thermal Bioeffects

Ultrasound waves can also have non-thermal interactions with tissues, with cavitation being the most significant interaction from a safety perspective. Stable cavitation has been associated with reversible increased permeability of the vascular endothelium. Inertial cavitation can lead to tissue heating and the formation of shock waves that can disrupt tissue integrity. Bubbles can exist in soft tissues for these processes to occur, and it is very difficult to induce inertial cavitation in soft tissues in the absence of bubbles. Bubbles/air can be introduced exogenously through the administration of ultrasonic contrast agents or may exist endogenously, especially in the lungs and large bowel. The FDA implements a cavitation related safety metric for diagnostic scanners called the Mechanical Index (MI), that evaluates the potential for cavitation. The MI is defined as

$$M I = \frac{p - .3}{\sqrt{f}} ,$$

where p−0.3 represents the peak in situ negative pressure (MPa) of the ultrasound wave measured in water with a hydrophone and assuming an acoustic attenuation of 0.3 dB/cm/MHz along the propagation path, and f represents the frequency (MHz) of the ultrasound wave. As the expression indicates, the MI increases with decreasing frequency. For diagnostic scanners, the upper limit for the MI is 1.9, with values above that representing an increased risk for cavitation. This limit is based upon experimental observations in water when bubbles of the optimum diameter were pre-existing.

ARFI images can and often are generated using MI values <1.9; however, due to power supply nonlinearity on the system, higher instantaneous powers for shorter durations (100 μs) may achieve larger displacements and better SNR than lower power, longer duration (1 ms) excitations. The intentional inducement of cavitation in excised issues for therapeutic purposes has been attempted, and cavitation has not been observed with the ARFI sequences, system, and transducers described ehrein, as is consistent with the lack of endogenous air bubbles in these tissues. The presence of needles and injected anesthetic could introduce air bubbles into the ROE that may provide seeds for cavitation. As with a normal nerve block procedure, great care will be taken to ensure that air is not present in the needle or injectate when performing the procedure. Air bubbles that are visible on standard B-mode imaging during the procedure will be avoided during ARFI imaging.

The presence of bubbles for an ARFI imaging acquisition may still be considered to be of minimal risk because any potential cavitation would be occurring in a region of soft tissue already being disrupted by needle piercing and the infusion of, e.g., up to 30 cc of fluid. ARFI imaging may not be performed if the region of interest contains lung for lower brachial plexus blocks.

Although certain embodiments according to the present invention are described herein with respect to ARFI imaging techniques, it should be understood that embodiments according to the invention can employ any suitable imaging technique, including B-mode images with or without concurrent ARFI images, to identify a decorrelation region of decorrelated data that is decorrelated between at least two image data sets as described herein.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. An ultrasound system for identifying a presence of injected fluid in a region of interest, the system comprising:
a controller configured to obtain first and second image data sets of a region of interest from an ultrasound transducer array; and
a decorrelation module configured to identify a decorrelation region of decorrelated data that is decorrelated between the first and second image data sets, wherein the decorrelation region indicates a presence of injected fluid in the region of interest.

2. The ultrasound system of claim 1, wherein the controller is configured to provide an image of the region of interest that identifies the decorrelation region.

3. The ultrasound system of claim 1, wherein the decorrelation module is configured to identify decorrelation data between the first and second image data sets based on a correlation coefficient (ρ) between a first signal, s₁(t), of the first image data set and a second signal, s₂(t), of the second data set as follows:
$$ρ = \frac{〈 {s}_{1} ( t ) {s}_{2}^{*} ( t ) 〉}{\sqrt{〈 {s}_{1} ( t ) {s}_{1}^{*} ( t ) 〉 〈 {s}_{2} ( t ) {s}_{2}^{*} ( t ) 〉}}$$
where * denotes the complex conjugate of the signal, ρ is a complex number whose magnitude denotes the similarity, or overall correlation, of the first and second signals and whose phase represents the phase difference between the first and second signals.

4. The ultrasound system of claim 3, wherein the decorrelation module is configured to identify decorrelation data such that ρ is less than about 0.98.

5. The ultrasound system of claim 3, wherein the signals s₁(t) and s₂(t), correspond to a region of image data for the first and second data sets, respectively.

6. The ultrasound system of claim 1, wherein the decorrelation module is configured to identify decorrelation data between three or more image data sets, and the controller is configured to provide an image of the region of interest that identifies combined decorrelation data from three or more image data sets.

7. An ultrasound method for identifying a presence of injected fluid in a region of interest, the method comprising:
obtaining first and second image data sets of a region of interest from an ultrasound transducer array; and
identifying a decorrelation region of decorrelated data that is decorrelated between the first and second image data sets, wherein the decorrelation region indicates a presence of injected fluid in the region of interest.

8. The ultrasound method of claim 7, further comprising providing an image of the region of interest that identifies the decorrelation region.

9. The ultrasound method of claim 8, further comprising injecting a fluid in the region of interest.

10. The ultrasound method of claim 9, further comprising modifying an injection location based on the image of the region of interest and the identified decorrelation region.

13. The ultrasound method of claim 7, wherein identifying decorrelation data between the first and second image data sets is based on a correlation coefficient (ρ) between a first signal, s₁(t), of the first image data set and a second signal, s₂(t), of the second data set as follows:
$$ρ = \frac{〈 {s}_{1} ( t ) {s}_{2}^{*} ( t ) 〉}{\sqrt{〈 {s}_{1} ( t ) {s}_{1}^{*} ( t ) 〉 〈 {s}_{2} ( t ) {s}_{2}^{*} ( t ) 〉}}$$
where * denotes the complex conjugate of the signal, ρ is a complex number whose magnitude denotes the similarity, or overall correlation, of the first and second signals and whose phase represents the phase difference between the first and second signals.

14. The ultrasound method of claim 13, wherein the decorrelation data is identified based on regions having ρ that is less than about 0.98.

15. The ultrasound method of claim 13, wherein the signals s₁(t) and s₂(t), correspond to a region of image data for the first and second data sets, respectively.

16. The ultrasound method of claim 7, further comprising identifying decorrelation data between three or more image data sets, and providing an image of the region of interest that identifies combined decorrelation data from three or more image data sets.

17. A computer program product for identifying a presence of injected fluid in a region of interest comprising:
a computer readable medium having computer readable program code embodied therein, the computer readable program code comprising:
computer readable program code configured to obtain first and second image data sets of a region of interest from an ultrasound transducer array; and
computer readable program code configured to identify a decorrelation region of decorrelated data that is decorrelated between the first and second image data sets, wherein the decorrelation region indicates a presence of injected fluid in the region of interest.

18. The computer program product of claim 17, further comprising computer readable program code that is configured to provide an image of the region of interest that identifies the decorrelation region.

19. The computer program product of claim 17, wherein computer readable program code that is configured to identify decorrelation data between the first and second image data sets is configured to identify decorrelation data based on a correlation coefficient (ρ) between a first signal, s₁(t), of the first image data set and a second signal, s₂(t), of the second data set as follows:
$$ρ = \frac{〈 {s}_{1} ( t ) {s}_{2}^{*} ( t ) 〉}{\sqrt{〈 {s}_{1} ( t ) {s}_{1}^{*} ( t ) 〉 〈 {s}_{2} ( t ) {s}_{2}^{*} ( t ) 〉}}$$
where * denotes the complex conjugate of the signal, ρ is a complex number whose magnitude denotes the similarity, or overall correlation, of the first and second signals and whose phase represents the phase difference between the first and second signals.

20. The computer program product of claim 13, wherein the computer readable program code that is configured to identify decorrelation data is configured to identify decorrelation data such that ρ is less than about 0.98.
